(19) United States
(12) Patent Application Publication
(10) Pub. No.: US 2003/0215487 A1
(43) Pub. Date: Nov. 20, 2003

(54) Matrix-type device for the transdermal delivery of testosterone applied to the non-scrotal skin

(75) Inventors: Dong-Yeun Kim, Seoul (KR); Dong-Woo Park, Seoul (KR); Sookyun Kim, Suwon (KR)

Correspondence Address: EDWARDS & ANGELL, LLP, P.O. BOX 9169, BOSTON, MA, 02209, US

(73) Assignee: IL YANG PHARM CO., LTD. Republic of Korea

(21) Appl. No.: 10/150,143

(22) Filed: May 17, 2002

(51) Int. Cl. A61K 9/70; A61K 31/57

(52) U.S. Cl. 424/449; 514/177

(57) ABSTRACT

The present invention relates to a matrix-type device for the transdermal delivery of testosterone comprising (i) a matrix layer containing a) a therapeutically effective amount of testosterone or the salt thereof, b) an adhesive polymer, and c) a penetration enhancer comprising N-methylpyrrolidone and oleoylsarcosine, and, (ii) a backing layer of polyurethane. The device of the present invention is applicable to a non-scrotal skin as compared with a known matrix-type device for the transdermal delivery and has an excellent skin-adhesion and has less tendencies of crystallizing testosterone.

### FIELD OF THE INVENTION

The present invention relates to a matrix-type device for the transdermal delivery of testosterone.

### BACKGROUND OF THE INVENTION

Testosterone, a male sex hormone, is related to a secondary sexual characteristic and sexual capability, and helps a protein synthesis and bone growth, and thus is essential for man. Deficiency of testosterone occurs in 1% of man of 20 to 50 years. The primary cause of testosterone deficiency is age, and other causes are diabetes, cancer, renal insufficiency, AIDS, and the like. Deficiency of testosterone may lead to fatigue, low vitality, myasthenia, melancholia, impotency and reduced sexual power, and so, testosterone therapy is needed for testosterone deficiency.

Traditional formulations for administering testosterone are tablet, injection, pellet, and the like. Oral formulations of testosterone such as tablet are broadly metabolized through the gastrointestinal tract and liver, and have a short half-life to take 2 or 3 times a day. Muscular injection or pellet of testosterone has a problem of accompanying pain when administrated. Researches for transdermal administration of testosterone have been made to resolve these problems. An advantage of transdermal administration of a drug over other administration routes is to avoid first-pass effect that is degradation and inactivation of a drug in the gastrointestinal tract and the liver. As a result, the side effect by undesired metabolites can be avoided by increased bioavailability of a drug, and desired effective concentration of a drug can be obtained with a small dose and fewer administration. Transdermal administration of a drug also has other advantage that deviation within and among patients may be minimized and drug concentration may be constantly maintained and so efficacy of the drug may be enhanced.

However, transdermal administration of testosterone is not so easy since a permeability of testosterone to the skin is very low, and testosterone is readily crystallized at the time of mixing and drying step. To resolve these problems, a reservoir-type device for transdermal delivery of testosterone in which testosterone is sealed in the form of alcohol-based gel has been developed and generally used. The reservoir-type device is contacted with the skin through microporous membrane and a strong adhesive is usually used to attach the device to the skin. However, the adhesive may cause a skin irritation. Alcohol oozed out through microporous membrane may also cause skin irritation.

The reservoir-type device is usually thick and thus causes uncomfortableness when applied to the skin such as back, shoulder, and the like. The manufacturing cost of reservoir-type device is relatively high compared with a matrix-type device for transdermal delivery of testosterone.

The matrix-type device in which testosterone is dispersed in the polymer matrix, is relatively thin and the manufacturing cost is relatively low compared with the reservoir-type device. However, the matrix-type device has such problems as crystallization and low solubility of testosterone, and the like and researches for a new matrix-type device have been steadily conducted.

Testoderm®, developed by Alza, is a matrix-type device for transdermal delivery of testosterone and is applied to the scrotal skin, and two forms of dose, 4 mg or 6 mg a day are on the market. However, Testoderm® has some problems such as:

(i) Permeability of testosterone is low,

(ii) Application to under the scrotum may cause irritation and the degree of compliance of patients due to inconvenience of attachment and detachment is low.

U.S. Pat. No. 5,152,997 discloses a reservoir-type device comprising a reservoir containing testosterone, skin permeation enhancer, and gelling agent, and means for maintaining the reservoir. Glycerol monooleate and methyl laurate are used as a skin permeation enhancer. The device is applied to the nonscrotal skin and a permeation rate of testosterone to the skin is relatively high. However, the device has such shortcomings as high cost by complex manufacturing processes, high possibility of skin irritation, bulkiness when applied to the skin, and the like since the device is a reservoir type.

U.S. Pat. No. 6,132,760 discloses a device for the transdermal delivery of testosterone wherein a series of terpene comprising pinene, d-limonene, carene, terpineol, terpinen-4-ol, carveol, carvone, pulegone, menthone, menthol, neomenthol, thymol, camphor, bomeol, citral, ionone and cineolea, and a derivative of C₆₋₂₀ fatty acid are used as a delivery enhancing adjuvant promoting transdermal delivery of testosterone. However, a skin permeation rate by the above delivery enhancing adjuvant is not sufficiently high, and testosterone tends to be crystallized during storage.

A conventional permeation enhancer used in the device for transdermal delivery of a drug includes propylene glycol monolaurate, polyoxyethylene oleylether, diethyltoluamide, dimethyl sulfoxide, dimethyl acetamide, dimethyl formamide, diethylene glycol monoethyl ether, N-alkyl pyrrolidone, alkylene glycol derivatives, azacycloalkanes, fatty acid and their esters, fatty acid alkanol amides, polyethyleneglycol derivatives, surfactants, alcohols, azones, terpenes and the like. It is known that mixing and using specific adjuvants may cause synergy. However, a need for a new permeation enhancer with a satisfying skin permeation rate still exists.

On the other hand, the device for transdermal delivery of testosterone often uses polyethylene and polypropylene of impermeable materials as a backing layer. However, both polyethylene and polypropylene have low permeability to the air and so when applied to the skin long time, the device is easily detached from the skin due to the sweat. Also, both polyethylene and polypropylene have little flexibility and so the device could not be applied to the skin of flexible region.

Non-woven fabrics that are easily permeable to the air is not suitable for a backing layer since their surface is not smooth, and another adhesive layer should be inserted between a non-woven fabric and a drug-containing adhesive layer, which increases the manufacturing cost and those two adhesive layers are mixed to block dissolution of a drug.

The present inventors have continuously researched a new matrix-type device for transdermal delivery of testosterone to resolve the above-mentioned problems, and found out that a device, which comprises a permeation enhancer comprising N-methylpyrrolidone and oleoylsarcosine, and a polyurethane backing layer, has an excellent permeation rate of testosterone to the skin enough to apply to the nonscrotal skin, better skin-adhesion, less irritation to the skin, and less tendency of crystallizing testosterone, and complete the present invention.

### DISCLOSURE OF THE INVENTION

The present invention provides a matrix-type device for transdermal delivery of testosterone comprising (i) a matrix layer containing a) a therapeutically effective amount of testosterone or the salt thereof, b) an adhesive polymer, and c) a penetration enhancer comprising N-methylpyrrolidone and oleoylsarcosine, and (ii) a backing layer of polyurethane.

Testosterone is present in the adhesive layer in a therapeutically effective amount, i.e., an amount effective to allow the device to deliver sufficient testosterone to achieve a desired therapeutic result in the treatment of a condition. The amount that constitutes a therapeutically effective amount varies according to the condition being treated, any drugs being coadministered with testosterone, desired duration of treatment, the surface area and location of the skin over which the device is to be placed, and the selection of adjuvant and other components of the transdermal delivery device. Accordingly, it is not practical to enumerate particular preferred amounts but such can be readily determined by those skilled in the art with due consideration of these and other appropriate factors. Generally, however, testosterone is present in the matrix layer in an amount of about 2 to 5% by weight based on the total weight of the matrix layer.

The matrix layer of the invention further comprises a penetration enhancer that enhances transdermal delivery of testosterone. Preferred penetration enhancers further comprise diethyltoluamide and/or sorbitan monooleate in addition to N-methylpyrrolidone and oleoylsarcosine. The total amount of the penetration enhancer will generally be about 2 to about 40 percent by weight based on the total weight of the matrix layer.

In certain preferred embodiments of the invention, the penetration enhancers comprise N-methylpyrrolidone, oleoylsarcosine, diethyltoluamide, and sorbitan monooleate in a weight ratio of 7-9:2-4:2-4:2-4. It has been found that combination of N-methylpyrrolidone, oleoylsarcosine, diethyltoluamide and sorbitan monooleate can most improve the skin permeability of testosterone or its salts.

The penetration enhancer of the present invention enables the penetration rate of testosterone to be more than 4.0 μg/cm²/hr, and so the device of the present invention may be applied to the nonscrotal skin, such as arm, thigh, abdomen and the like. Further, the penetration enhancer used in the device of the present invention helps to solubilize testosterone in the matrix without a solvent, and so, the device according to the present invention has simple manufacturing procedures compared to the previous device in which testosterone should be solubilized in a separate solvent and mixed with a penetration enhancer and an adhesive.

The above matrix layer containing the drug may optionally further comprise pharmaceutically acceptable additives like dissolution adjuvants, etc., for example, propyleneglycol monolaurate, polyoxyethylene oleylether, ethyl alcohol, isopropyl alcohol, dimethylsulfoxide, oleoyl alcohol, dimethylacetamide, dimethyl formamide, polyethylene glycol, propylene glycol, polyoxyethylene ester and alkanol amines.

Polyurethane used as a backing layer in the device of the present invention has great skin adhesive strength and flexibility enough for the device to be applied to the curved skin, and great air permeability to be attached with less sweat when the device is applied and less irritability to the skin but penetration to the skin is relatively low in comparison with polyethylene and polypropylene. However, since the penetration enhancer contained in the matrix which greatly enhances the skin penetration rate of testosterone or its salts, polyurethane possessing the above-mentioned advantages can be used as a backing layer material in the device of the present invention.

Further, the polyurethane backing layer serves to inhibit crystallization of testosterone as proved in Test Example 4 below, and thus maximize testosterone concentration within the device to enable the present device to be freely applied to the nonscrotal skin.

The polyurethane used in the device according to the present invention preferably has a moisture permeability of 500 to 5000 g/m².hr when measured under Japanese Industry Standard (JIS) Z-0208 which provides that the moisture permeability is measured by placing the sample on a cup filled with 100 g of distilled water for 24 hours at 40° C. and 90% relative humidity. A preferred polyurethane backing layer has a flexibility of 2 to 10 folds.

The device of the present invention comprises a release liner, if necessary. The release liner used in the present invention is prepared by a conventional method, for example, coating a thin layer of polyester with fluoropolymer. The release liner serves to protect the matrix layer containing the drug before the application of the device to the skin.

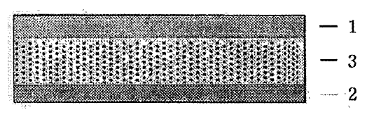

The device of the present invention comprising the release liner is shown in FIG. 1. One surface of the adhesive layer (3) containing the active component is contacted with the release liner(2), and the other surface of the adhesive layer (3) is contacted with the backing layer (1).

The adhesive polymer contained in the matrix layer of the device according to the present invention is a conventionally used material in a device for transdermal delivery of a drug. A preferred adhesive layer is a pressure sensitive adhesive that may be mixed with testosterone such as polyacrylate, rubber, or silicon adhesive. An example of the adhesive polymer used in the device of the present invention is Durotak® 87-2852.

The matrix layer containing the drug may be multiple layered, if necessary. If the matrix layer containing the drug is manufactured as multiple layers, compositions of each layer may be formulated to control the release rate of the active component. For example, if the matrix layer is 3-layered, the nearest layer to the skin is formulated to release the drug most rapidly, and the most distant layer is formulated to release the drug most slowly.

The device of the present invention may be prepared by a conventional preparation method as follows: testosterone or the salt thereof is mixed with dissolution adjuvant, penetration enhancer, and the like to obtain a solution or suspension, and the solution or the suspension is added to an adhesive polymer with stirring for 20 to 30 minutes, and leaving for 20 to 60 minutes and then removing the air bubbles. The obtained mixture is applied to a release paper coated with silicon at the thickness of 100 to 250 μm and dried for 10 minutes at 80 to 110° C. to obtain a matrix layer. The matrix layer is then covered with a polyurethane backing layer at the thickness of 20 to 500 μm.

The following examples further illustrate the present invention. These examples are not intended to limit the invention in any manner.

### EXAMPLE 1

2.0 g of testosterone, 4.0 g of N-methylpyrrolidone(NMP) and 1.5 g of olcoylsarcosine were combined and stirred for 30 minutes in a container. After testosterone was completely solubilized, 50 g of Durotak® 87-2852 (National Starch Co. Ltd.) was added into the container and the container was then shaken for 2 hours. The resulting formulation was coated onto a silicone release liner at the thickness of 200 μm. The coated liner was oven dried for 5 minutes at 80° C. The coated liner was then laminated to a polyurethane backing layer (MSP-1000, available from Dongsung Chemical Co. Ltd, Pusan, Korea) at the thickness of 200 μm. The laminate was die-cut into 70 cm² patches.

| Formulation | Weight |
|---|---|
| Testosterone | 2.0 g |
| N-methylpyrrolidone | 4.0 g |
| Oleoylsarcosine | 1.5 g |
| Durotak ® 87-2852 | 50.0 g |
| Total | 57.5 g |

### EXAMPLE 2

Patches were prepared by the same method described in Example 1 using the following formulation:

| Formulation | Weight |
|---|---|
| Testosterone | 2.0 g |
| N-methylpyrrolidone | 4.0 g |
| Oleoylsarcosine | 3.0 g |
| Durotak ® 87-2852 | 50.0 g |
| Total | 59.0 g |

### EXAMPLE 3

Patches were prepared by the same method described in Example 1 using the following formulation:

| Formulation | Weight |
|---|---|
| Testosterone | 2.0 g |
| N-methylpyrrolidone | 4.0 g |
| Oleoylsarcosine | 1.5 g |
| Diethyltoluamide (DEET) | 1.5 g |
| Durotak ® 87-2852 | 50.0 g |
| Total | 59.0 g |

### EXAMPLE 4

Patches were prepared by the same method described in Example 1 using the following formulation:

| Formulation | Weight |
|---|---|
| Testosterone | 2.0 g |
| N-methylpyrrolidone | 4.0 g |
| Oleoylsarcosine | 1.5 g |
| Sorbitan monooleate | 1.5 g |
| Durotak ® 87-2852 | 50.0 g |
| Total | 59.0 g |

### EXAMPLE 5

Patches were prepared by the same method described in Example 1 using the following formulation:

| Formulation | Weight |
|---|---|
| Testosterone | 2.0 g |
| N-methylpyrrolidone | 4.0 g |
| Oleoylsarcosine | 1.5 g |
| Diethyltoluamide (DEET) | 1.5 g |
| Sorbitan monooleate | 1.5 g |
| Durotak ® 87-2852 | 50.0 g |
| Total | 60.5 g |

### Test Example 1

Patch samples were prepared in accordance with the same method as described in Example 1 except that compositions of the penetration enhancers are presented in Table 1 below. Skin Penetration Test and Crystallization Test were conducted for these patch samples.

(1) Skin Penetration Test

Skin Penetration Test using a Franz-cell which is generally used for evaluation of a transdermal therapeutic system was conducted as follows:

A hairless mouse 4 weeks old of 20 to 25 g body weight was suffocated to death in the ether decicator and was fixed on an operating table. A 3×3cm² of abdominal skin was then carefully separated. The separated skin was fixed again so that its inner skin was exposed. Subcutaneous fat, tissues, blood vessels, and the like were carefully removed from the skin. The external surface of the obtained skin was completely dried, and the patch was applied to the skin and pressed to make uniform contact with the skin. The resulting patch/skin laminate was placed patch side up across the orifice of the lower portion of the above diffusion cell. The diffusion cell was assembled and the lower portion was filled with receptor fluid of 40% polyethylene glycol 400 (PEG400) so that the receptor fluid was in contact with the skin. The receptor fluid was stirred using a magnetic stirrer. The sampling port was covered except when in use. The cell was then placed in a chamber of constant temperature of 32° C. The receptor fluid was stirred at the rate of 600 rpm by means of a magnetic stirrer throughout the experiment to make the sample uniform sample and the diffusion barrier reduced on the dermal side of the skin. 0.5 ml of receptor fluid was withdrawn at 3, 6, 10, 18, 24 and 30 hours later and immediately replaced with fresh fluid. The withdrawn fluid was then analyzed for testosterone using the high performance liquid chromatography. The flux rate was calculated. The penetration test was conducted for 3 samples of each test formulations. The average flux rates are shown in the Table 1.

<Operation Conditions for the HPLC>

| | |
|---|---|
| Column: | Cosmosil 5C₁₈ (5 μm, 4 mm × 150 mm, Nacalai Tasque, |
| | Japan) |
| Mobile Phase: | acetonitrile/50 mM acetate buffer (pH 4.0), 40:60 |
| Detector: | UV, 242 nm |
| Flow rate: | 1.0 ml/min |

(2) Crystallization Test

The same patches used in the above test (1) were stored for 4 months at 25° C. After 4 months, crystallization of each sample patch was observed. Crystallization levels were evaluated into 5 grades of 0 to 5.

0: no crystallization during storage

5: much crystallization during storage

the results are shown in Table 1.

TABLE 1
Comparison of skin penetration enhancing
effects according to penetration enhancers.

| Experiment | Penetration | Skin Penetration | Crystallization |
|---|---|---|---|
| No. | enhancer (weight: g) | rate (μg/cm²/hr) | level |
| 1 | NMP (4) | 0.65 ± 0.18 | 0 |
| 2 | NMP (4) + | 1.48 ± 0.22 | 1 |
| | Capric acid (3) | | |
| 3 | NMP (4) + | 0.79 ± 0.10 | 1 |
| | Oleic acid (3) | | |
| 4 | NMP (4) + | 0.44 ± 0.04 | 2 |
| | Lauric acid (3) | | |
| 5 | NMP (4) + | 0.51 ± 0.14 | 0 |
| | Lauryl alcohol (3) | | |
| 6 | NMP (4) + | 4.12 ± 0.24 | 0 |
| | Oleoylsarcosine (3) | | |
| 7 | NMP (4) + | 2.61 ± 0.14 | 1 |
| | Tricaprin (3) | | |
| 8 | NMP (4) + | 2.76 ± 0.48 | 0 |
| | Lauroylsarcosine (3) | | |

As shown in Table 1, the penetration enhancer of Experiment 6, NMP+oleoylsarcosine, exhibited the most potent skin penetration rate and no crystallization. The patch containing more than 3 g of oleoylsarcosine exhibited skin irritation (the data are not shown).

### Test Example 2

Patch samples containing the penetration enhancers presented in Table 2 below were prepared by the same method as described in Example 1. Skin Penetration Test and Crystallization Test were conducted for these patch samples by the same method as described in Test Example 1. Results of the skin penetration test and the crystallization test are shown in Table 2.

TABLE 2
Comparison of skin penetration enhancing
effects according to penetration enhancers

| | | Skin | Crystal- |
|---|---|---|---|
| Experiment | Penetration | Penetration rate | lization |
| No. | enhancer (weight: g) | (μg/cm²/hr) | level |
| 9 | NMP(4) + | 2.53 ± 0.31 | 0 |
| | Lauroylsarcosine (1.5) + | | |
| | Sorbitan monooleate (1.5) | | |
| 10 | NMP (4) + | 4.27 ± 0.44 | 0 |
| | Lauroylsarcosine (1.5) + | | |
| | Azone (1.5) | | |
| 11 | NMP (4) + | 4.54 ± 0.43 | 0 |
| | Oleoylsarcosine (1.5) + | | |
| | Sorbitan monooleate (1.5) | | |
| 12 | NMP (4) + | 2.01 ± 0.19 | 0 |
| | Oleoylsarcosine (1.5) + | | |
| | Azone (1.5) | | |
| 13 | NMP (4) + | 0.83 ± 0.22 | 0 |
| | Polyethyleneglycol 200 | | |
| | monolaurate (1.5) + | | |
| | Sorbitan monooleate (1.5) | | |
| 14 | NMP (4) + | 2.17 ± 0.69 | 1 |
| | Capric acid (1.5) + | | |
| | Sorbitan monooleate (1.5) | | |
| 15 | NMP (4) + | | |
| | Capric acid (1.5) + | 2.51 ± 0.72 | 1 |
| | Oleoylsarcosine (1.5) | | |
| 16 | NMP (4) + | 4.03 ± 0.96 | 0 |
| | Oleoylsarcosine (1.5) + | | |
| | Lauroylsarcosine (1.5) | | |
| 17 | NMP (4) + | 2.61 ± 0.11 | 0 |
| | Oleic acid (1.5) + | | |
| | Oleoylsarcosine (1.5) | | |
| 18 | NMP (4) + | 1.98 ± 0.83 | 0 |
| | Oleic acid (1.5) + | | |
| | Lauroylsarcosine (1.5) | | |
| 19 | NMP (4) + | 4.12 ± 0.24 | 0 |
| | Oleoylsarcosine (3) | | |

As shown in Table 2, the penetration enhancers of Experiments 10 and 11 that consist of 3 components exhibited more excellent skin penetration enhancing effects than the penetration enhancer of Experiment 19 that consists of 2 components.

### Test Example 3

Patch samples containing the penetration enhancers presented in Table 2 below were prepared in accordance with the same method as described in Example 1. Skin Penetration Test and Crystallization Test were conducted for these patch samples by the same method as described in Test Example 1. Results of the skin penetration test and the crystallization test are shown in Table 3.

TABLE 3
Comparison of skin penetration enhancing
effects according to penetration enhancers

| | | Skin | Crystal- |
|---|---|---|---|
| Experiment | Penetration | Penetration rate | lization |
| No. | enhancer (weight: g) | (μg/cm²/hr) | level |
| 20 | NMP(4) + | 3.30 ± 0.70 | 0 |
| | Lauroylsarcosine (1.5) + | | |
| | Sorbitan monooleate (1.5) + | | |
| | DEET (1.5) | | |
| 21 | NMP (4) + | 3.92 ± 0.69 | 0 |
| | Lauroylsarcosine (1.5) + | | |
| | Oleoylsarcosine (1.5) + | | |
| | Sorbitan monooleate (1.5) | | |
| 22 | NMP (4) + | 5.12 ± 0.92 | 0 |
| | Oleoylsarcosine (1.5) + | | |
| | Sorbitan monooleate (1.5) + | | |
| | DEET (1.5) | | |
| 23 | NMP (4) + | 2.90 ± 0.44 | 0 |
| | Oleoylsarcosine (1.5) + | | |
| | Azone (1.5) + | | |
| | Sorbitan monooleate (1.5) | | |
| 24 | NMP (4) + | 4.06 ± 0.29 | 0 |
| | Polyethyleneglycol 200 | | |
| | monolaurate (1.5) + | | |
| | Sorbitan monooleate (1.5) + | | |
| | Oleoylsarcosine (1.5) | | |
| 25 | NMP (4) + | 2.50 ± 0.90 | 0 |
| | DEET (3) + | | |
| | Oleoylsarcosine (1.5) | | |
| 26 | NMP (4) + | 4.69 ± 0.66 | 0 |
| | Oleoylsarcosine (3) + | | |
| | Lauroylsarcosine (1.5) | | |
| 27 | NMP (4) + | 4.79 ± 0.83 | 0 |
| | Oleic acid (1.5) + | | |
| | Oleoylsarcosine (3) | | |
| 28 | NMP (4) + | 3.11 ± 0.46 | 0 |
| | Oleoylsarcosine (1.5) + | | |
| | Lauroylsarcosine (3) | | |

As shown in Table 3, the penetration enhancers of Experiment 22, 24, 26 and 27 exhibited excellent skin penetration enhancing effects, i.e., the skin penetration rates were more than 4. However, the penetration enhancers of Experiment 24, 26 and 27 exhibited skin side effects (the data are not shown). Thus, it has been found that the penetration enhancer of Experiment 22, the combination of NMP, oleoylsarcosine, sorbitan monooleate and DEET is the most suitable penetration enhancer for transdermal delivery of testosterone.

### Test Example 4

Patch samples containing 3.0 g of oleic acid and 4.0 g of NMP and having a backing layer as polyurethane(MSP-1000, available from Dongsung Chemical Co. Ltd, Pusan, Korea) were prepared by the same method described in Example 1.

Patch samples having polypropylene backing layer (Cotran® 9722, available from 3M Co. Ltd.) and polyethylene backing layer (Cotran® 9722, available from 3M Co. Ltd.), respectively, were prepared by the same method.

Observation of crystal formation was conducted for these patch samples at the interval of 1 month by the same method as described in Test Example 1(2). Results of the crystallization test are shown in Table 4.

TABLE 4

Crystallization level

| | After | After | After | After |
|---|---|---|---|---|
| | 1 month | 2 month | 3 month | 4 month |
| Patch using the PU | 0 | 0 | 0 | 0 |
| backing film | | | | |
| Patch using the PE | 0 | 1 | 4 | 5 |
| backing film | | | | |
| Patch using the PP | 0 | 1 | 4 | 5 |
| backing film | | | | |

Note:
PU = polyurethane
PE = polyethylene
PP = polypropylene

As shown in Table 4, a crystallization level during storage are varied depending on materials of the backing layer under the same conditions. In particular, testosterone in the patches having the PE or PP backing layer began crystallizing after 2 months whereas testosterone in the patches having the PU backing layer is not crystallized even after 4 months. Thus, it has been found that the PU backing layer are the most suitable for the device of transdermal delivery according to the present invention. In conclusion, testosterone of high concentration may be contained in the device of the present invention.

### Test Example 5

Plasma concentrations of testosterone were measured in test mice after application of the patch samples of Example 4 and Androderm®, a known reservoir-type Testosterone patch.

First, 8 male white mice having the body weight of about 220 g were fasted for 16 hours and fixed on a fixing table. Polyethylene tube (PE50, Clay Adams®, available from Becton Dickinson, N.J., USA) was inserted into the right carotid artery of each mouse under ether anesthesia. After the mice came out from the anesthetic, 1 patch of Androderm (containing 12.2 mg of testosterone) and 1 patch of Example 4 (containing 20.0 mg of testosterone/35cm²), respectively, were applied to the abdomens of the 4 hairless mice. 0.25 ml of the blood was withdrawn at the time of 0.5, 1, 2, 3, 4, 6, 8, 10 and 12 hours later and immediately replaced with the same volume of heparin-Na solution (50 unit/ml). The withdrawn blood was immediately centrifuged at 10,000 rpm for 5 minutes to separate the plasma. The obtained plasma was then analyzed for the concentration of testosterone using the high performance liquid chromatography (available from Shiseido, Japan).

<Operation Conditions for the HPLC>

| | |
|---|---|
| Column: | A column: Capcelpak MF column (4.6 mm × 50 mm) |
| | B column: Capcelpak C18, (1.5 mm × 250 mm) |
| Mobile Phase: | A column: acetonitrile/50 mM acetate buffer (pH 4.0), |
| | 20:80 |
| | B column: acetonitrile/50 mM acetate buffer (pH 4.0), |
| | 40:60 |
| Detector: | UV, 242 nm |
| Flow rate: | A: 0.6 ml/min, B: 0.1 ml/min |

Average cumulative plasma concentrations are shown in FIG. 2. As shown in FIG. 2, the cumulative plasma concentration of testosterone of the device of the present invention, a matrix-type device, was similar to that of Androderm, a reservoir-type device.

### Test Example 6

6 patch samples prepared according to Example 4 and 6 patches of Androderm® were applied to the back skin of 12 healthy men 25 to 40 years old, respectively. The skin irritation level was observed at 12, 24 and 48 hours later. The results are shown in Table 5.

TABLE 5
Results of the skin irritation tests.

| | Skin Irritation Level | | | | | | | | | | | |
|---|---|---|---|---|---|---|---|---|---|---|---|---|
| | Androderm | | | | | | Patch sample of Example 4 | | | | | |
| 12 hr | 0 | 0 | 0 | 0 | 0 | 0 | 0 | 0 | 0 | 0 | 0 | 0 |
| 24 hr | 1 | 1 | 0 | 1 | 0 | 0 | 0 | 0 | 0 | 0 | 0 | 0 |
| 48 hr | 2 | 1 | 0 | 2 | 0 | 3 | 1 | 0 | 0 | 0 | 1 | 0 |

Skin Irritation Level:
0 No change
1 Mild red spots
2 Moderate red spots or mild swelling
3 Severe red spots or swelling

As shown in Table 5, 3 subjects to which Androderm was applied, began exhibiting skin irritation after 24 hours, and showed moderate or severe skin irritation after 48 hours. However, in case of the subjects to which the patch sample of Example 4 was applied, no subjects exhibited skin irritation after 24 hours, and 2 subjects showed mild skin irritation after 48 hours. Therefore, it has been found that the patch of the present invention has less side effects of skin irritation than the known patch.

The device of the present invention is applicable to non-scrotal skin contrary to the known matrix-type device for transdermal delivery and has excellent skin-adhesion and less tendency of testosterone crystallization.

What is claimed is:

1. A matrix-type device for transdermal delivery of testosterone comprising
(i) a matrix layer containing
a) a therapeutically effective amount of testosterone or the salt thereof,
b) an adhesive polymer, and
c) a penetration enhancer comprising N-methylpyrrolidone and oleoylsarcosine, and,
(ii) a backing layer of polyurethane.

2. The device according to claim 1 wherein the penetration enhancer further comprises diethyltoluamide and/or sorbitan monooleate.

3. The device according to claim 1, further comprising a release liner.

4. The device according to claim 1 or 2 wherein the testosterone or the salt thereof is in an amount of 2 to 5% of weight based on the total weight of the matrix layer.

5. The device according to claim 1 or 2 wherein the penetration enhancer is in an amount of 2 to 40% of weight based on the total weight of the matrix layer.

6. The device according to claim 1 or 2 wherein the polyurethane backing layer has a moisture permeability of 500 to 5000 g/m².24 hr.

7. The device according to claim 6 wherein the polyurethane backing layer has a flexibility of from 2 to 10 folds.

8. The device according to claim 1 wherein thickness of the matrix layer has a thickness of from 20 to 500 μm and the backing layer has a thickness of from 30 to 500 μm.
